SEQUENCE LISTING (11 sequences)

SEQ ID NO: 1 - DNA, 20 bp, Artificial
  Mouse TGase 2 sense strand
ccaagcaaaaccgcaaactg

SEQ ID NO: 2 - DNA, 24 bp, Artificial
  Mouse TGase 2 antisense strand
tgatggctctcctcttaccctttc

SEQ ID NO: 3 - DNA, 23 bp, Artificial
  Mouse iNOS sense strand
actaccagatcgagccctggaac

SEQ ID NO: 4 - DNA, 24 bp, Artificial
  Mouse iNOS antisense strand
gcaagctgagaggctgctcccagg

SEQ ID NO: 5 - DNA, 22 bp, Artificial
  Double-stranded consensus oligonucleotide for NF-kB
agttgaggggactttcccaggc

SEQ ID NO: 6 - protein, 5 aa, Artificial
  pro-elafin sequence
DPVKG

SEQ ID NO: 7 - protein, 8 aa, Artificial
  pro-elafin/antiflammin sequence
KVLDGQDP

SEQ ID NO: 8 - DNA, 20 bp, Artificial
  Rat TNF-a sense
ccccattactctgacccctt

SEQ ID NO: 9 - DNA, 20 bp, Artificial
  Rat TNF-a antisense
aggcctgagacatcttcagc

SEQ ID NO: 10 - DNA, 21 bp, Artificial
  Rat B-actin sense
ggcattgtaaccaactgggac

SEQ ID NO: 11 - DNA, 19 bp, Artificial
  Rat B-actin antisense
tgttggcatagaggtcttt

(19) United States
(12) Patent Application Publication
(10) Pub. No.: US 2006/0286608 A1
(43) Pub. Date: Dec. 21, 2006

(54) Method for screening transglutaminase 2 inhibitor or activator

(76) Inventor: Soo-Youl Kim, Seoul (KR)

Correspondence Address: ARENT FOX PLLC, 1675 BROADWAY, NEW YORK, NY, 10019, US

(21) Appl. No.: 11/245,539

(22) Filed: Oct. 7, 2005

(30) Foreign Application Priority Data
Jun. 17, 2005 (KR) 10-2005-0052619

(51) Int. Cl. G01N 33/567; G01N 33/53

(52) U.S. Cl. 435/7.2

(57) ABSTRACT

Disclosed herein is a method for screening a TGase 2 inhibitor or activator, based on the finding that TGase 2-induced NF-κB activation is attributed to the polymerization of I-κBα, in which the level of free or polymerized I-κBα proteins or the level of NF-κB is measured to determine the TGase 2 inhibitor or activator.

### CROSS-REFERENCE TO RELATED APPLICATION

This application claims priority under 35 U.S.C. § 119 to Korean Patent Application No. 10-2005-0052619 filed Jun. 17, 2005, the entire specification claims and drawings of which are incorporated herewith by reference.

### BACKGROUND OF THE INVENTION

1. Field of the Invention

The present invention relates to a method for screening for a TGase 2 inhibitor or activator.

2. Description of the Prior Art

Transglutaminase 2 (TGase 2, E.C. 2.3.2.13, protein-glutamine γ-glutamyltransferase; TGase 2) belongs to a family of Ca²⁺-dependent enzymes that catalyze N^{ε}-(γ-_{L}-glutamyl)-_{L}-lysine isopeptide bond formation between peptide bound lysine and glutamine residues. N^{ε}-(γ-_{L}-glutamyl)-_{L}-lysine cross-linking stabilizes intra- and extracellular proteins as marcromolecular assemblies that are used for a variety of essential physiological purposes, such as barrier function in epithelia, apoptosis, and extracellular matrix formation. TGase 2 is normally expressed at low levels in many different tissues and is inappropriately activated in a variety of pathological conditions. Particularly, it is known that TGase 2 level increases in inflammatory diseases.

In a previous study conducted by the present inventors, it was demonstrated that the TGase expression increased under lipopolysaccharide (LPS) treatment in BV-2 microglia, and that the release of nitric oxide (NO) is dramatically reduced by TGase inhibitors. During the LPS-induced microglia activation, TGase activity increased about 5-fold in microglia after 24 hours of exposure to LPS in a time-dependent manner. This suggests that the increase of NO synthesis is associated with the increase of TGase 2 expression (Park et al., (2004) Biochem. Biophys. Res. Commun. 323, 1055 1062). However, although LPS is revealed to induce TGase expression and thus the synthesis of NO, which plays an important role in immune responses such as inflammation, the precise mechanism by which TGase 2 increases NO synthesis so as to induce immune responses still remains unclear.

### SUMMARY OF THE INVENTION

Leading to the present invention, intensive and thorough research, conducted by the present inventors, into the mechanism of TGase 2 in immune responses, resulted in the finding that TGase 2 induces the polymerization of inhibitory subunit α of nuclear factor-κB (I-κBα), resulting in a loss in affinity for nuclear factor-κB (NF-κB), so that NF-κB is activated to bring about an inflammation. Based on this finding, TGase 2 inhibitors or activators can be screened by measuring the level of the I-κBα protein, the degree of polymerization of the I-κBα protein, or the activity of NF-κB in accordance with the present invention.

One object of the present invention is to provide a method for screening for a Transglutaminase 2 (TGase 2) inhibitor or activator, comprising: (a) treating cells expressing I-κBα and NF-κB with a candidate inhibitor or activator of TGase 2; (b) inducing the expression of TGase 2 in the cells; and (c) comparing the level of free I-κBα, the level of polymerized I-κBα or the activation of NF-κB between the cells treated with the candidate inhibitor or activator and a control treated without the candidate inhibitor or activator.

Another object of the present invention is to provide a method for screening a TGase 2 inhibitor or activator, comprising: (a) treating isolated I-κBα with a candidate inhibitor or activator of TGase 2; (b) treating the isolated I-κBα with isolated TGase 2; and (c) detecting the level of free or polymerized I-κBα.

### BRIEF DESCRIPTION OF THE DRAWINGS

FIG. 1 shows TGase 2 induction in LPS-induced BV-2 microglia.

FIG. 2 shows in vivo targets of TGase in the NF-κB cascade.

FIG. 3 shows the function of TGase 2 of depleting free I-κBα without ubiquitination, with the concomitant polymerization of I-κBα.

FIG. 4 shows the results of testing whether free or polymerized I-κBα binds to NF-κB.

FIG. 5 shows an increase in NF-κB activity and a decrease in I-κBα activity due to TGase transfection.

FIG. 6 shows the effect of TGase 2 on the cellular level of I-κBα.

FIG. 7 shows the effect of TGase 2 inhibitors on LPS-induced rat brain injury.

### DETAILED DESCRIPTION OF THE INVENTION

Although the expression and activity of TGase 2 increase upon immune responses, the precise mechanism by which TGase 2 induces immune responses has remained unclear.

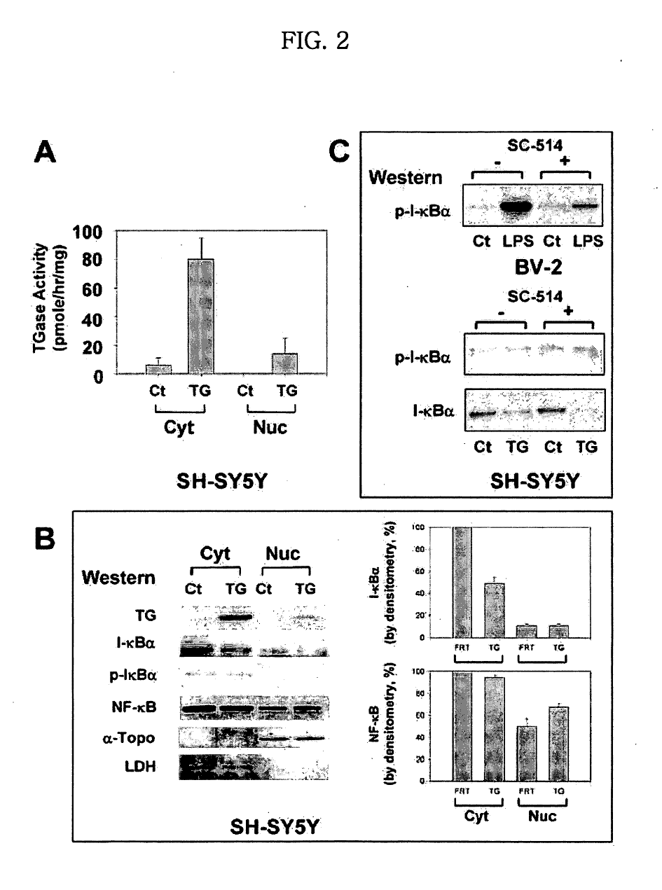

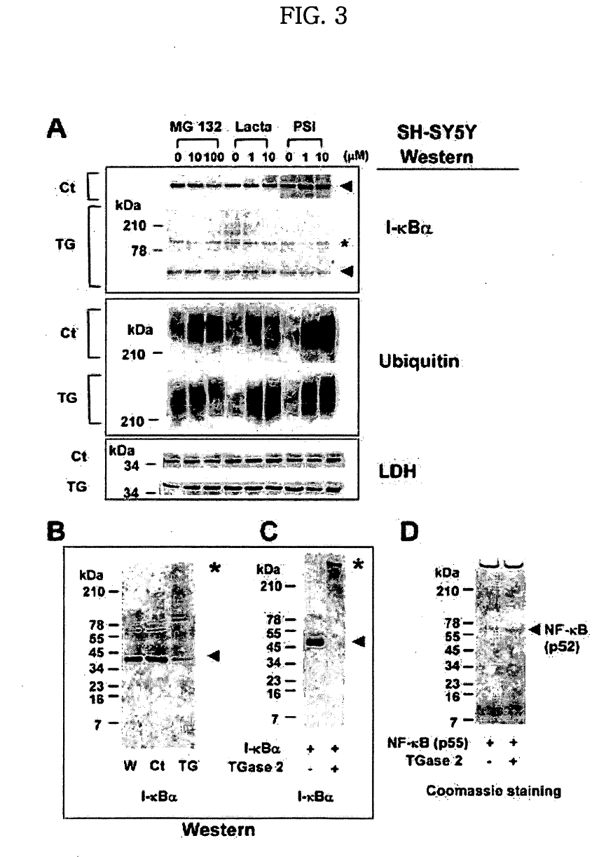

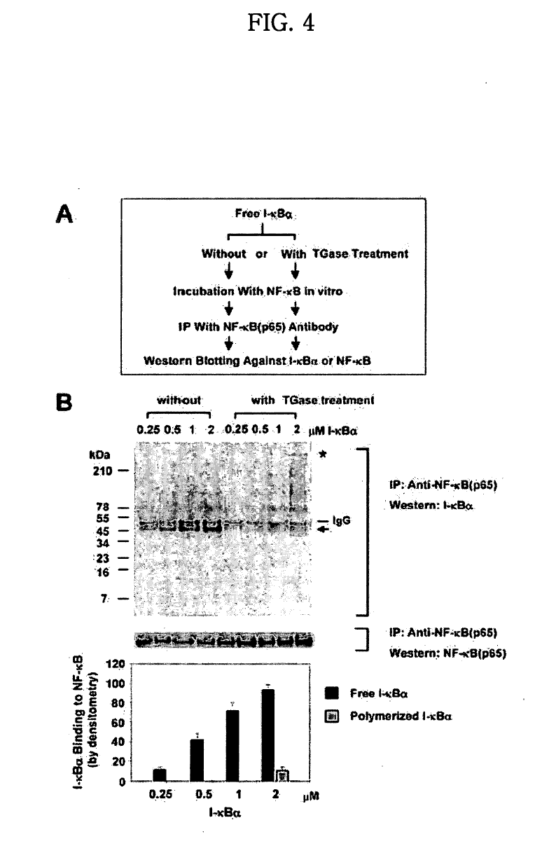

In the present invention, the mechanism in which TGase 2 induces an inflammation is discovered with the findings that TGase 2 activates NF-κB (FIG. 2) and the TGase 2-induced NF-κB activation results from the dissociation of I-κBα and NF-κB (FIG. 4) as TGase 2 induces I-κBα polymerization (FIG. 3). TGase 2 causes I-κBα to undergo polymerization, resulting in a decrease in cellular, free I-κBα level and an increase in cellular polymerized I-κBα level. Polymerized I-κBα loses its ability to bind to NF-κB. Indeed, densitometry analysis showed that the binding efficiency of polymerized I-κBα to NF-κB loses 90% or more of the level of free I-κBα. This mechanism is different from the previously suggested mechanism in which NF-κB is activated by the phosphorylation and degradation of I-κBα.

As the present inventors revealed that TGase 2 induces I-κBα polymerization, which consequently activates NF-κB, the understanding and control of the immune response mechanism through TGase 2 become feasible.

With this understanding, controllers of TGase 2 activity can be detected by measuring the level of free I-κBα proteins, the level of polymerized I-κBα proteins, or the degree of activation of NF-κB. Since TGase 2 greatly varies in activity with even a small change in calcium concentration because it is a calcium-dependent enzyme, reliable results can be preferably achieved by measuring the level of free I-κBα proteins, the level of polymerized I-κBα proteins, or the degree of activation of NF-κB, rather than by measuring the level of TGase 2 proteins.

In accordance with one embodiment of the present invention, a method for screening for a Transglutaminase 2 (TGase 2) inhibitor or activator, comprising: (a) treating cells expressing I-κBα and NF-κB with a candidate inhibitor or activator of TGase 2; (b) inducing the expression of TGase 2 in the cells; and (c) comparing the level of free I-κBα, the level of polymerized I-κBα, or the activation of NF-κB between the cells treated with the candidate inhibitor or activator and a control not treated with the candidate inhibitor or activator, is provided.

The term “inhibitor” as used herein means a material that acts to reduce TGase 2 expression or activity. The term “activator” as used herein means a material that increases TGase 2 expression or activity.

The term “candidate inhibitor” or “candidate activator” is a material that is expected to be an inhibitor or an activator of TGase 2, respectively. As these candidates, single compounds, such as organic or inorganic compounds, macromolecules, such as proteins, carbohydrates, nucleic acid molecules (RNA, DNA, etc.) and lipids, and composites composed of plural compounds may be included.

As used herein, the term “treatment” implies that a candidate, that is, a TGase 2 candidate inhibitor or candidate activator is brought into direct contact with TGase 2, and the material acts on a cell membrane so that a signal generated from the cell membrane transfers to TGase 2. Therefore, the candidate materials must be understood to include materials incapable of penetrating cell membranes as well as material capable of penetrating cell membranes. At this time, the candidate materials are treated within the range of effective amounts. Herein, the term “effective amount” means an amount sufficient to induce a reaction, and since no accurate results are obtained outside the range of effective amounts, inhibition or activation must be analyzed within the effective amount range.

For screening for TGase 2 inhibitors or activators, all TGase 2-expressing cells, originating from humans or animals, such as cows, goats, pigs, mice, rabbits, hamsters, rats, guinea pigs, etc., whether primary, secondary, or immortalized cells, may be used. Alternatively, a cell which is manipulated with a TGase 2 gene-carrying recombinant vector to over-express TGase 2 stably or transiently therein can be used. Preferable are nervous system-originated cells known to express TGase 2 at a low level. In the present invention, a BV-2 strain originated from microglia, or SH-SY5Y originated from neuroblastoma cells is used and manipulated to over-express TGase 2 stably or transiently therein.

The screening of TGase 2 inhibitors or activators can be conducted using experimental animals, such as mice, rabbits, rats, guinea pigs, etc., in vivo as well as at a cellular level.

A predetermined time period after being treated with a candidate inhibitor or activator of TGase 2, cells that express I-κBα and NF-κB may be induced to express TGase 2. Alternatively, cells expressing I-κBα and NF-κB may be induced concurrently with the treatment with a candidate inhibitor or activator of TGase 2. Also, if necessary, the induction of TGase 2 expression may be conducted in advance of the treatment with a candidate inhibitor or activator of TGase 2.

The expression of TGase 2 may be induced by any factor that is known to induce TGase 2 expression, for example UV light, ionizing radiation, glutamate, calcium ionophore, maitotoxin, RA (retinoic acid), inflammation-inducible cytokines, oxidative environment, viral infection, etc., and the factors and methods for inducing TGase 2 expression are not specifically limited.

The degree of inhibition or activation of TGase 2 by treatment with a candidate material can be significantly detected by comparing an I-κBα level, a polymerized I-κBα level, or NF-κB activity with that of a control.

Treatment with a TGase 2 activator increases the cellular level of polymerized I-κBα, enhances the activity of NF-κB and decreases the cellular level of free I-κBα significantly when compared with a control. In contrast, the cellular level of polymerized I-κBα and the activity of NF-κB decrease, resulting from being increased the cellular level of free I-κBα in the case of treatment with a TGase 2 inhibitor.

To detect the level of free or polymerized I-κBα, a specific antibody against I-κBα may be used. Antigen-antibody complexes formed are quantitatively compared between cells treated with and without candidates. Absolute or relative differences in the amount of antigen-antibody complexes formed can be determined through molecular biological or histochemical assays, which are exemplified by immunoprecipitation, immunostaining, Western blotting, immunochemical staining, immunofluorescent staining, etc., but are not limited thereto. Preferable is a Western blotting assay, which can be performed by, for example, separating proteins extracted from cell through SDS-PAGE, and reacting the proteins with an anti-I-κBα antibody so as to determine levels of free and polymerized I-κBα through the pattern and strength of bands.

In the detection methods, the amounts of antigen-antibody complexes formed can be quantitatively analyzed by measuring the signal intensity of a detection label.

The term “detection label” as used herein means a composition detectable by a spectroscopic, photochemical, biochemical, immunochemical, chemical, physical, or other appropriate means. Examples of detection labels useful in the present invention include enzymes, fluorescent materials, ligands, luminescent materials, microparticles, redox molecules, and radioactive isotopes, but are not limited thereto.

NF-κB activation can be detected using a reporter assay or EMSA. The detection of the cellular level of a reporter protein linked to a promoter having an NF-κB binding site leads to the measurement of NF-κB activation. As a reporter protein for the detection of NF-κB activation, an enzyme, such as β-galactosidase, alkaline phosphatase, acetylcholine esterase, glucose oxidase, luciferase, phosphofructokinase, phosphoenolpyruvate carboxylase, aspartate aminotransferase, phosphoenolpyruvate decarboxylase, or β-lactamase, may be used. The activity of a reporter protein can be measured by detecting the fluorescence or chemoluminescence emitted after reaction with a substrate or using an assay method, such as Northern blotting, Western blotting, RNase protection assay, etc. In the present invention, a SEAP (secreted alkaline phosphatase) reporter system 3 (pNFkB-SEAP; BD Biosciences Clontech) was employed to assay NF-κB activation.

Alternatively, or in combination therewith, NF-κB activation may also be analyzed using EMSA (Electrophoretic Mobility Shift Assay). After nuclear extracts from cells are reacted with a labeled oligonucleotide having an NF-κB binding site, the association of the oligonucleotide with NF-κB is detected to measure the activity of the NF-κB transcriptional factor. In the present invention, EMSA was performed with the [³²P]ATP-labeled oligonucleotide of SEQ. ID. NO. 5.

In addition, when isolated I-κBα or TGase 2 is used to detect inhibitors or activators in vitro, inhibitors or activators that can directly interact with TGase 2 can be screened. Therefore, in accordance with another embodiment, the present invention provides a method for screening a TGase 2 inhibitor or activator, comprising: (a) treating isolated I-κBα with a candidate inhibitor or activator of TGase 2; (b) treating the isolated I-κBα with isolated TGase 2; and (c) detecting the level of free or polymerized I-κBα.

In the present invention, the term “isolated” used herein with respect to protein, means substantially free of other proteins, that are present in the natural source of the macromolecule. The isolated protein contains less than 20% (by dry weight) of contaminating protein, and more preferably less than 5% of contaminating protein. Isolation techniques for proteins expressed in cells are not specifically limited in the present invention.

The inhibitors or activators, which are screened not in vivo, but in vitro, are materials reacting directly to TGase 2.

The isolated I-κBα, treated with a TGase 2 candidate inhibitor or activator, may be reacted with isolated TGase 2 simultaneously or sequentially at different times. Also, if necessary, the isolated I-κBα and NF-κB may be reacted, and then the candidate inhibitor or activator may be added.

The level of free or polymerized I-κBα proteins can be detected as described above.

Furthermore, the inhibitors screened using the method described above can be used to inhibit the TGase 2-associated NF-κB cascade, thereby effectively treating or preventing diseases related to an increase of TGase 2 activity, such as inflammatory diseases or cancer.

Generally, inflammatory diseases are divided into autoimmune diseases and neurodegenerative diseases.

Autoimmune diseases are closely associated with aberrant activation of T cells and macrophages, which causes serious inflammation. Abnormal increases of TGase 2 expression were reported in autoimmune inflammatory myopathies and celiac diseases (Choi et al., (2000) J. Biol. Chem. 275, 88703-88710; Choi et al., (2004) Eur. Neurol. 51, 10-14; Bruce et al., (1985) Clin. Sci. 68, 573-579). An increased level of TGase 2 was found in autoimmune diseases as a result of macrophage activation, and the increase of TGase 2 expression seems to be closely associated with autoantibody formation (Novogrodsky et al., (1978) Proc. Natl. Acad. Sci. U.S.A. 75, 1157-1161; Murtaugh et al., P. J. (1983) J. Biol. Chem. 258, 11074-11081; Leu et al., (1982) Exp. Cell Res. 141, 191-199). Examples of autoimmune diseases related to the overexpression or overactivation of TGase 2 include celiac disease (Dieterich et al., (1997) Nat. Med. 3, 797-801), dermatitis herpetiformis (Dieterich, et al., (1999) J Investig. Dernatol. 113, 133-136), type 1 diabetes (Lampasona et al., (1999) Diabetologia 42, 1195-1198), Lupus (Sanchez, et al., (2000) J Autoimmun. 15, 441-449), and Rheumatoid Arthritis (Picarelli et al., (2003) Clin. Chem. 49, 2091-2094), but are not limited thereto.

The activation of microglial cells that produce neurotoxic factors, such as nitric oxide (NO) and TNF-α, is known to be associated with brain inflammation (Minagar et al., (2002) J. Neurol. Sci. 202, 13-23; Catania et al., (1998) Ann. N.Y. Acad. Sci. 856, 62-68). The synthesis and release of these factors constitute part of the innate immunity that enables the host to destroy invading pathogens. However, when nitric oxide (NO) is synthesized and accumulated excessively, it acts as a cause of neurodegeneration (Liu et al., (2002) Ann. N.Y. Acad. Sci. 962, 318-331). Particularly, TGase 2 induced in activated astrocytes is known to be involved in the mechanism generating neurodegenerative diseases (Campisi et al., (2003) Brain Res. 978, 24-30; Monsonego et al., (1997) J. Biol. Chem. 272, 3724-3732). Examples of the neurodegenerative diseases related to the overexpression or overactivation of TGase 2 include Parkinson's disease (Junn et al., (2003) Proc. Natl. Acad. Sci. U.S.A 100, 2047-2052; Andringa et al., (2004) FASEB J 18, 932-934), Alzheimer's disease (Kim et al., (1999) J Biol. Chem. 274, 30715-30721; Citron et al., (2001) J. Biol. Chem. 276, 3295-3301), and neuro-AIDS (Roberts et al., (2003) Am. J. Pathol. 162, 2041-2057), but are not limited thereto.

Cyclooxygenase-2 (COX-2) is a target gene that is typically induced by NF-κB. Now, COX-2 is regarded as important in the prevention and treatment of cancer as well as in the treatment of inflammation. In cancer cells and malignant tumor tissues, an increase in COX-2 expression is induced to produce a far greater amount of prostaglandin than in normal cells (Kargman et al., (1995) Cancer Research, 55:2556-2559; Ristimaki et al., (1997) Cancer Research, 57:1276-1280). Functioning to promote angiogenesis and cell proliferation, prostaglandins, such as prostaglandin E₂ (PGE₂), can provide environments suitable for the growth of cancerous cells when they are produced in excess. Furthermore, the overexpression of COX-2 is known to restrain apoptosis and enhance cancer metastasis. Additionally, an increase of COX-2 expression was confirmed in various cancers, and COX inhibitors are reported to reduce the occurrence of cancers (Noguchi et al., (1995) Prostaglandins, Leukotrienes, and Essential Fatty Acids, (1997) 53:325-329; Thompson et al., (1997) Cancer Research, 57:267-271). Consequently, selective COX-2 inhibitors can be used as anticancer agents as well as anti-inflammatory agents.

Based on the fact that COX-2 expression is induced by TGase 2, TGase 2 inhibitors can be used as anticancer agents. Examples of cancers that can be therapeutically treated using the TGase 2 inhibitors screened in accordance with the present invention include large intestinal cancer, small intestinal cancer, rectal cancer, anal cancer, esophageal cancer, pancreatic cancer, stomach cancer, kidney cancer, uterine carcinoma, breast cancer, lung cancer, lymphoma, thyroid cancer, prostatic carcinoma, leukemia, skin cancer, colon cancer, encephaloma, bladder cancer, ovarian cancer, and gallbladder cancer, but are not limited thereto.

In addition, the activators obtained by the method in accordance with the present invention can be used to promote TGase 2-associated signal transduction within cells, thereby effectively treating or preventing diseases related to a decrease in TGase 2 activity, such as diseases due to viral infection.

TGase 2 expression is known to increase with RA (retinoic acid) (Moore et al. (1984) J Biol Chem 259, 12794-12802). RA is also known to help inhibit viral infection or enhance immune responses, thereby contributing to the treatment of diseases (Lotan R. (1996) FASEB J. 10, 1031-109). Accordingly, TGase 2-induced NF-κB activation plays an important role in the defense against viral infection. As well known to those skilled in the art, immune activity depends on the activity of NF-κB, and NF-κB can be activated by TGase 2 overexpression. Thus, the administration of the activators screened by the method in accordance with the present invention induce TGase 2-associated signal transduction so as to effectively treat or prevent viral infection diseases.

A better understanding of the present invention may be obtained in light of the following examples which are set forth to illustrate, but are not to be construed to limit the present invention.

### EXAMPLE 1

### Microglia Activation by LPS

Murine BV-2 cells exhibit phonotypic and functional properties of reactive microglial cells. The BV-2 cells were grown and maintained in DMEM (Dulvecco's modified Eagle's medium) (Invitrogen) supplemented with 10% FCS (fetal calf serum) and penicillin/streptomycin at 37° C. in a humidified incubator under 5% CO₂. To activate BV-2, the cells were treated with LPS (100 μg/ml; Sigma) for 24 hours. After LPS treatment for 24 hours with or without inducible nitric-oxide synthase(iNOS) inhibitor, 0, 50, and 100 μM N^{G}-monomethyl-_{L}-arginine(L-NMMA) (Sigma), proteins were extracted with radioimmunoprecipitation assay buffer (1× phosphate-buffered saline (PBS), 1% Nonidet P-40, 0.5% sodium deoxycholate and 0.1% SDS) containing protease inhibitors from BV-2 harvest, followed by analysis for TGase 2 activity.

Nitric Oxide Measurement

Accumulated nitric oxide was measured in the cell supernatant after LPS treatment for 24 hours by Griess reaction. A 200 μl aliquot of the cell supernatant in each well of a 96-well microtiter plate was mixed with 100 μl of the Griess reagent [1% sulfanilamide (Fluka), 0.1% naphthylethylenediamine dihydrochloride (Fluka), 2.5% H₃PO₄], and the absorbance was read at 540 nm using a plate reader.

Semi-Quantitative RT-PCR of Mouse TGase 2 and iNOS

Semi-quantitative RT-PCR was performed using competitive mimic templates as internal controls. To prepare total RNA for RT-PCR, the cells were lysed with a TRIzol reagent. Samples of the total RNA were reverse-transcribed at 42° C. using the first strand synthesis kit (Promega) with avian myeloblastosis virus reverse transcriptase, and PCR was performed for the transcripts of iNOS and TGase 2 using corresponding specific primer sets. For each PCR, 1.5 mM MgCl₂, 200 μM dNTP, 0.2 μM of each primer, 0.5 unit Taq polymerase, and a predetermined amount of a template were contained in a volume of 20 μl. The mimic templates of TGase 2 and iNOS were constructed by PCR. The mimics of mouse TGase 2 and mouse iNOS were prepared from 2014-2338 bp and 1451-2043 bp, respectively. RT-PCR products thus obtained were 526 bp for target TGase 2, 345 bp for mimic TGase 2, 593 bp for target iNOS, and 345 bp for mimic iNOS. For the RT-PCR, a primer set of SEQ. ID. NOS. 1 and 2, and a primer set of SEQ. ID. NOS. 3 and 4 were used:

| | |
|---|---|
| Mouse TGase 2 sense strand | |
| 5′-CCAAGCAAAACCGCAAACTG-3′ | (SEQ. ID. NO. 1) |
| Mouse TGase 2 antisense strand | |
| 5′-TGATGGCTCTCCTCTTACCCTTTC-3′ | (SEQ. ID. NO. 2) |
| Mouse iNOS sense strand | |
| 5′-ACTACCAGATCGAGCCCTGGAAC-3′ | (SEQ. ID. NO. 3) |
| Mouse iNOS antisense strand | |
| 5′-GCAAGCTGAGAGGCTGCTCCCAGG-3′ | (SEQ. ID. NO. 4) |

Stable Transfection of TGase 2

The human neuroblastoma cell line SH-SY5Y used for transfection was obtained from the ATCC (American Type Culture Collection). SH-SY5Y cells were grown in DMEM/Ham's F12 medium (50:50) supplemented with 10%-heat inactivated fetal bovine serum, glutamine, and penicillin/streptomycin. To avoid clonal variation, the Flp-In™ System (Invitrogen, Co) was employed. SY5Y/TG cells, which carry a pcDNA5/FRT vector containing a full-length human TGase 2 gene, were adopted and SH-SY5Y cells carrying an empty vector were used as a control. After selection, the apoptosis of SH-SY5Y/TG cells was found not to be increased through the criteria of normal cell growth, LDH (lactate dehydrogenase) release, 4′,6′-diamidino-2-phenylindole, dihydrochloride staining, caspase activity, and annexin V staining. This coincides with the previous report that TGase 2-transfected neuroblastoma cells do not show increased apoptosis unless they are subjected to oxidative stress.

To examine whether the effect of TGase 2 on cellular targets can be reversed, a tetracycline-induced expression system using the EcR 293 cell line (Flp-In T-Rex-293; Invitrogen) was employed. After the introduction of a pcDNA5/FRT carrying a full length human TGase 2 into the EcR 293 cell and selection with hygromycin, TGase 2 was induced by treatment with 1 μg/ml of tetracycline for 24 hours in DMEM supplemented with 10% FBS.

IKK Inhibitor Treatment

To examine whether TGase 2-induced NF-κB activation is IKK-dependent, the IKK-2 inhibitor SC-514 (Calbiochem) was employed. As a positive control, BV-2 was activated with LPS with or without SC-514. Before 30 min of LPS induction, BV-2 was pretreated with or without 10 μM SC-514 for 1 hour. Also, SH-SY5Y and SH-SY5Y/TG cells were treated with or without 10 μM SC-514 for 1 hour. Following cell harvest, cytosolic fractions were collected for Western blotting analysis.

TGase Activity Assay

Enzymatic activity was determined using a modified TGase assay method for measuring the incorporation of [1,4-¹⁴C] putrescine into succinylated casein.

Western Blotting

The cytosolic fractions were prepared using a nuclear extract kit (Sigma). The samples were separated from 10-20% gradient SDS gels in Tricine buffer (Invitrogen) and then transferred onto a polyvinylidene difluoride membrane (Invitrogen). Western blotting was conducted as established previously. Antibodies to NF-κBp65, I-κBα, phospho-IκB-α (Ser32), I-κB kinaseβ(IKK-β), phospho-IKKα (Ser180)/IKKβ(Ser181), and NF-κB activating kinase were obtained from Cell Signaling Technologies (Beverly, Mass.). Antibodies to NIK, IKKα, and α-topoisomerase I were obtained from Santa Cruz Biotechnology (Santa Cruz, Calif.). Antibodies to LDH (Research Diagnostics, Inc., Flanders, N.J.), ubiquitin (Sigma), and TGase 2 (clone CUB 7402; NeoMarkers, Union City, Calif.) were purchased as indicated. The concentrations of primary and secondary antibodies were 5 and 0.1 μg/ml, respectively. The blot was then developed by ECL (enhanced chemiluminescence) (Pierce, Milwaukee, Wis.). To determine the purity of extracted cytosolic and nuclear fractions, anti-LDH and anti-α-topoisomerase were used for the cytosolic fraction and the nuclear fraction, respectively.

In Vitro Cross-Linking Experiments

The full-length human I-κBα was cloned into a pET-30 Ek/LIC vector (Novagen) through PCR using full-length I-κBα cDNA (pCMV-IκBα; BD Biosciences), expressed and purified through a HisTrap column (Amersham Biosciences). A human recombinant NF-κB(p52) protein was obtained from Santa Cruz Biotechnology. I-κBα (2 μM) or NF-κB (p52) (2 μM) was incubated with or without 0.001 unit of guinea pig liver TGase 2 for 30 min at 37° C. in 20 μl of Tris-HCl (pH7.5) containing 10 mM CaCl₂. After the incubation, the sample was analyzed by Western blotting for I-κBα and by Coomassie protein staining for NF-κB(p52).

Binding Efficiency of Free and Polymerized I-κBα to NF-κB

The full-length I-κBα was prepared as described above. The full-length human NF-κB (p65) was obtained from Active Motif Co. Incubation of 2 μM I-κBα with TGase 2 (0.001 unit) for 30 min at 37° C. showed the complete polymerization of I-κBα (FIG. 3C). To examine the binding efficiency of free or polymerized I-κBα to NF-κB (p65), various concentrations of I-κBα (0.25-2.0 μM)were incubated with or without TGase 2 (0.001 unit) for 30 min at 37° C. in 50 mM Tris-Cl buffer, pH 7.5, containing 10 mM CaCl₂, and the reaction was terminated by the addition of 20 mM EDTA. NF-κB(2 μM) was treated with the I-κBα mixture for 1 hour at room temperature. For immunoprecipitation, the mixture was gently mixed with 5 μg of an NF-κB(p65) antibody for 1 hour at room temperature, and a protein A/G-agarose-conjugated slurry (Pierce) was added to the mixture which was subsequently allowed to stand for 1 hour at room temperature. After centrifugation at 2000×g for 5 min, the pellets thus obtained were boiled in a loading buffer, and were loaded on a 10-20% gradient Tricine-polyacrylamide gel. Following electrophoresis, proteins were transferred onto a polyvinylidene difluoride membrane for Western blotting analysis.

Transient Transfection of TGase 2

cDNAS encoding full-length human TGase 2 cloned into a pSG5 vector (Stratagene) were used to induce the expression of TGase 2. The transient transfection was performed using a calcium phosphate method. When mouse BV-2 cells were grown to 80% confluence in 6-well tissue culture dishes, the medium was replaced with 2 ml of a fresh culture medium. Plasmids (1 μg) were prepared in the presence of 25 μmol of calcium in 100 μl of a medium. An equal volume of 2× HEPES-buffered saline was prepared. The mixture of plasmid and calcium was added to the 2× HEPES-buffered saline buffer, and the resulting mixture was incubated for 20 min at room temperature and strongly vortexed and added dropwise to the culture medium.

NF-κB Activity Assay

NF-κB activity was measured using a SEAP (Secreted alkaline phosphatase) reporter system 3 (pNFkB-SEAP; BD Biosciences Clontech). At 12 hours after transient transfection, the culture medium was replaced with a fresh one. After 24 hours, the medium was collected for SEAP assay and the cells were harvested for β-galactosidase assay. The vehicle vector pSG5 (Stratagene) was used as a control. Cells treated with a pGAL plasmid (1 μg) were co-transfected with expression vectors that could be normally expressed in the β-galactosidase assay. The SEAP assay was carried out according to the protocol of the manufacturer (BD Biosciences Clontech). Values were the means of three measurements (S.D.<10%).

Activity Measurement of NF-κB Using EMSA (Electrophoretic Mobility Shift Assay)

Nuclear extracts of BV-2 microglia and SH-SY5Y were prepared from a non-transfected control, a vehicle control (pSG5; Stratagene), and TGase 2-transfected (pSG5/TG) cells using a nuclear extract kit (Sigma). A double-stranded consensus oligonucleotide for NF-κB (5′-AGT TGA GGG GAC TTT CCC AGG C-3′: SEQ. ID. NO. 5) was end-labeled with [³²P]ATP. Binding reactions containing equal amounts of the nuclear extract protein (6 μg) and 10 fmol (˜10,000 cpm; Cherenkov counting) of the oligonucleotide were performed for 30 min in a binding buffer (10 mM HEPES, pH 7.9, 50 mM KCl, 2 mM EDTA, 0.3 mg/ml bovine serum albumin, 6 mM MgCl₂, 10% glycerol, 1 mM dithiothreitol, 2 μg poly dI-dC). Total reaction volumes were held at 20 μl. Reaction products were separated on 6% polyacrylamide gels and analyzed using a bioimaging analyzer (Fuji).

Effect of TGase Inhibitors on Reduced I-κBα in SH-SY5Y/TG Cells

Cystamine is known to inhibit TGase activity by blocking the access of a glutamine residue in substrate proteins to the TGase active site. Iodoacetamide (Sigma) is also known to inhibit TGase activity as a strong competitive irreversible inhibitor. The effects of these TGase inhibitors were demonstrated in many studies. E2 (DPVKG: SEQ. ID. NO. 6) and R2 (KVLDGQDP: SEQ. ID. NO. 7) were designed to contain a pro-elafin sequence and a pro-elafin/antiflamnin sequence, respectively, therein. The effectiveness of R2 and E2 as TGase 2 inhibitors was previously demonstrated in vitro and in vivo. In order to examine the effect of TGase inhibitors on the decrease in I-κBα level, the SH-SY5Y/TG culture was treated with different inhibitors for 30 min, followed by the separation of the cytosolic fraction using a nuclear extract kit (Sigma).

Effect of TGase Inhibitors on LPS-Induced Rat Brain Injury

Male Sprague-Dawley rats (Samtako, Osan, Korea) weighing 190-220 g were used as experimental models for intraperitoneal LPS injection as described previously. All experimental procedures were approved by the Seoul National University Care of Experimental Animals Committee. A solution of LPS (2.5 mg/kg) in 0.9% saline or 0.9% sterile saline was intraperitoneally injected into rats. To determine the effect of TGase inhibitors, rats were intraperitoneally injected with an R2 peptide (25 μM), an E2 peptide (25 μM, and dexamethasome (1 mg/kg) at 30 min before and at the time of LPS injection. Dexamethasome injection was used as a positive control.

Immunohistochemistry

After 1 hour of intraperitoneal injection with LPS or saline, rats were anesthetized with 1% ketamine (30 mg/kg) and xylazine hydrochloride (4 mg/kg). Brains were perfused through the heart with saline containing 0.5% sodium nitrite and 10 units/ml heparin, followed by perfusion with 4% paraformaldehyde in PBS (0.1 M, pH 7.2). Brains were removed, rinsed with PBS, and cryoprotected in sucrose. Sections were prepared on a sliding microtome (40 μm) at the level of the subfornical organ. A monoclonal antibody (TG-100; NeoMarkers) to TGase 2 was used to subject TGase 2 to immunohistochemical staining. Brain sections were blocked with 1% BSA in PBS and incubated overnight with a primary antibody solution (1:200 dilution). After being washed for 30 min with PBS, the sections were incubated with biotinylated goat anti-mouse IgG for 1 hour, followed by incubation with peroxidase-avidin for 1 hour and then visualization with a Vector Elite Kit (Vector Laboratories, Burlingame, Calif.). Floating sections were mounted on slides, dehydrated with graded alcohols, and coverslipped. For controls for staining specificity, pre-absorption of a mixture of a primary TGase 2 antibody and purified guinea pig liver TGase 2 (Sigma), omission of the primary antibody; or the replacement of the primary antibody with nonimmune serum was employed.

Comparative RT-PCR

Samples of total RNA from rat brain tissues were reverse-transcribed by a first strand synthesis kit (Poche Molecular Biochemicals), and PCR was performed on the transcripts of TNF-α and β-actin. RT-PCR primers for targets were made from 923-1242 bp of TNF-α and 91-760 bp of rat β-actin. To ensure a linear relationship between amounts of PCR products and total RNA, variable numbers of PCR cycles were used. The PCR primer sequences were as follows:

| | |
|---|---|
| Rat TNF-α sense | |
| 5′-CCCCATTACTCTGACCCCTT-3′ | (SEQ. ID. NO. 8) |
| Rat TNF-α antisense | |
| 5′-AGGCCTGAGACATCTTCAGC-3′ | (SEQ. ID. NO. 9) |
| Rat β-actin sense | |
| 5′-GGCATTGTAACCAACTGGGAC-3′ | (SEQ. ID. NO. 10) |
| Rat β-actin antisense | |
| 5′-TGTTGGCATAGAGGTCTTT-3′ | (SEQ. ID. NO. 11) |

### EXAMPLE 2

### Induction of TGase 2 in LPS-Induced BV-2 Microglia

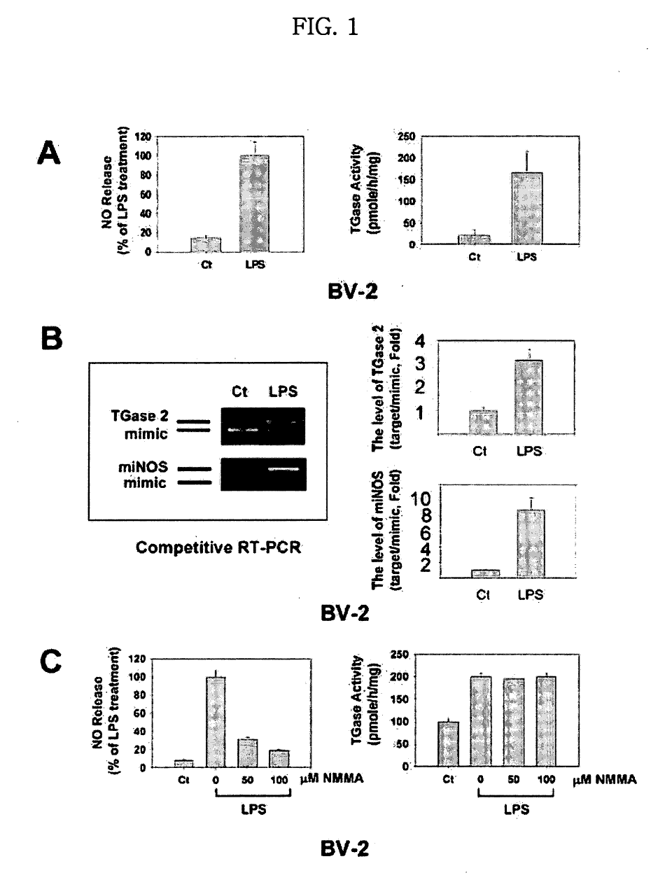

The expression of TGase 2 was increased by LPS in BV-2 microglia. After 24 hours of LPS treatment, the release of NO was increased 10-fold with a concomitant 5-fold increase in TGase 2 activity (FIG. 1A). RT-PCR analysis for iNOS and TGase 2 after treated BV-2 cells with LPS showed that TGase 2 was increased 3-fold concomitant with a 10-fold increase in iNOS (FIG. 1B). In addition, it was observed that the transient transfection of TGase 2 into the BV-2 microglia increases NF-κB activity. iNOS was previously reported to be triggered by NF-κB activation. Therefore, the data suggested that TGase 2 is probably involved in the regulation of the NF-κB cascade. To examine whether TGase 2 expression was regulated by NO, BV-2 cells were treated with LPS and then NMMA (iNOS inhibitor) (FIG. 1C). NMMA did not affect TGase activity, but reduced NO secretion in a dose-dependent manner.

### EXAMPLE 3

### In Vivo Target of TGase in NF-κB Cascade

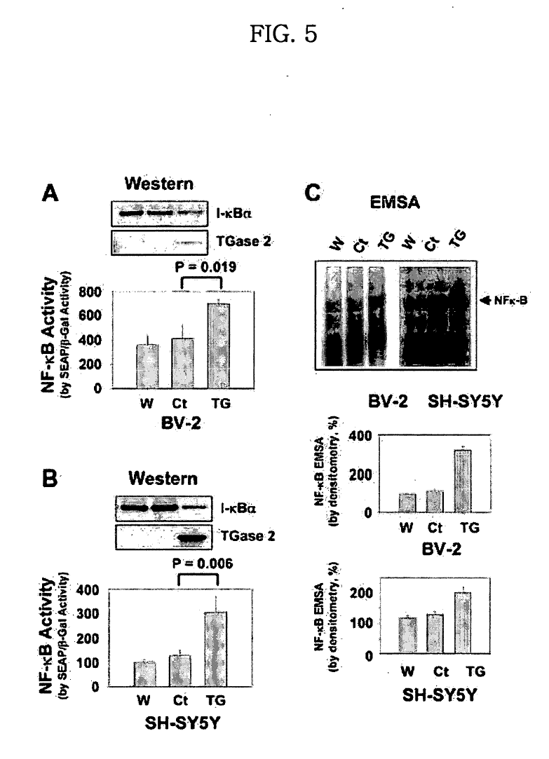

To identify targets of TGase in the NF-κB cascade, SH-SY5Y cells were stably transfected with TGase 2 and were subjected to Western blotting experiments (FIG. 2). TGase 2 activity was observed to increase 8-fold in the cytosolic fraction of the SH-SY5Y/TG cells (FIG. 2A). Further, Western blotting analyses exhibited no changes in the NF-κB activating kinase NIK IKKα, and p-IKK. When compared between SH-SY5Y and SH-SY5Y/TG cells, I-κBα was decreased 50% in the cytosol and NF-κB was increased 30% in the nucleus, and p-I-κBα was not changed (FIG. 2B). To examine whether the decrease in free I-κBα due to TGase 2 transfection was IKK-dependent or not, the IKK-2 inhibitor SC-514 was used for the treatment of the cells. As seen in FIG. 2C, SC-524 treatment did not change the level of p-I-κBα in SH-SY5Y and SH-SY5Y/TG cells whereas LPS-treated BV-2 cells showed a decrease in p-I-κBα with SC-514. This coincides with the experimental results that in TGase 2-overexpressed BV-2 cells, TGase 2 activity increased 5- or higher fold and I-κBα decreased as measured by Western blotting, as shown in FIG. 5A.

### EXAMPLE 4

### Polymerization of I-κBα by TGase 2 and Depletion of Free I-κBα without Ubiquitination

To examine whether TGase 2 reduces the level of I-κBα via a ubiquitin-proteasome system, SH-SY5Y/TG cells were incubated for 6 hours with proteasome inhibitors, such as MG132, lactacystin, or carbobenzoxy-_{L}-isoleucyl-gamma-ₜ-butyl-_{L}-alanyl-_{L}-leucinal (FIG. 3A). The cytosol was extracted from cells and was carried out Western blotting for I-κBα and ubiquitin. LDH activity in the medium and caspase-9 expression by Western blotting in the treated cells were not detected in the course of the experiment. If NF-κB expression induced by TGase 2 depends on the IKK/ubiquitin/proteasome pathway, the level of both I-κBα and ubiquitinated I-κBα should be increased. As seen in FIG. 3A, the level of I-κBα in SH-SY5Y/TG cells increased due to proteasome inhibition. Increased ubiquitinated I-κBα was not detected by Western blotting. Western blotting analysis showed a reduced level of I-κBα in SH-SY5Y/TG cells, which appears to be a result from the polymerization of I-κBα (FIG. 3B). The incubation of purified I-κBα with 0.001 unit of TGase 2 purified from a liver of guinea pig for 30 min resulted in completely polymerized I-κBα (FIG. 3C). The same polymerization was not observed upon the incubation of NF-κB(p52) with TGase 2 (FIG. 3D).

### EXAMPLE 5

### Binding of Free or Polymerized I-κBα to NF-κB

Binding probability of polymerized I-κBα with NF-κB was examined. Upon TGase 2 treatment as in FIG. 3C, free I-κBα was completely cross-linked to a high molecular weight polymer (FIG. 4). Free I-κBα was treated with or without TGase 2, followed by incubation with NF-κB. The mixture was immunoprecipitated using an NF-κB antibody, and the precipitates were subjected to Western blotting analysis against I-κBα. The free form of I-kB was detected to bind very effectively to NF-κB in a dose-dependent manner (FIG. 4B). In contrast, polymerized I-κBα was lost its binding ability.

### EXAMPLE 6

### NF-κB Activation by TGase 2 Transfection

NF-κB activation was analyzed using an NF-κB/SEAP reporter assay normalized to β-galactosidase activity and an EMSA with nuclear fractions after transfection with TGase 2. Western blotting of TGase 2 and I-κBα was performed. The transient transfection of TGase 2 into BV-2 cells, using cDNAs encoding full-length human TGase cloned in a pSG5 vector, reduced the level of I-κBα in the cytosol, resulting in a 2-fold increase in NF-κB activity (FIG. 5A). The stable transfection of TGase 2 in SH-SY5Y cells reduced the level of I-κBα in the cytosol, with a concomitant 3-fold or higher increase in NF-κB activity (FIG. 5B). Using a double-stranded concensus oligonucleotide for NF-κB end-labeled with [P³²]ATP, binding reactions were carried out with nuclear extracts from BV-2 and SH-SY5Y cells which were transfected with or without TGase 2 (FIG. 5C). Gel shift showed that the level of NF-κB increased 3- and 2-fold in BV-2 and SH-SY5Y cells, respectively, after TGase 2 transfection.

### EXAMPLE 7

### Effect of TGase 2 Expression on Level of I-κBα

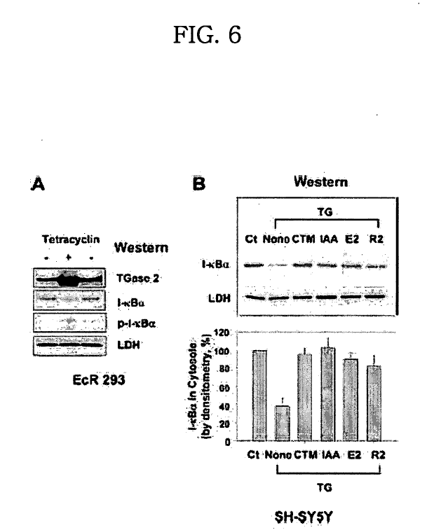

The effect of TGase 2 expression on the level of I-κBα was examined in EcR 293 and SH-SY5Y cells. To control TGase 2 expression, a tetracycline-induced expression system was applied to EcR 293 cell line (FIG. 6A). In FIG. 6A, EcR 293 cells were collected before incubation (left), after incubation in a medium containing 1 μg/ml of tetracycline for 24 hours (center), and after incubation in a medium containing 1 μg/ml of tetracycline for 24 hours and then in a fresh medium containing no tetracycline for an additional 24 hours (right). As seen in FIG. 6A, the expression of TGase 2 was found to reciprocally regulate the level of free I-κBα, but not the level of p-I-κBα. To examine whether TGase 2 inhibitors can result in the same effect, SH-SY5Y/TG cells were incubated for 30 min with a TGase inhibitor, such as cystamine, idoacetamide, E2 peptide, or R2 peptide. TGase inhibitors were found to reduce the cytosolic I-κBα level almost to the control level as measured by Western blotting analysis (FIG. 6B).

### EXAMPLE 8

### Effect of TGase 2 Inhibitor on LPS-Inducted Rat Brain Injury

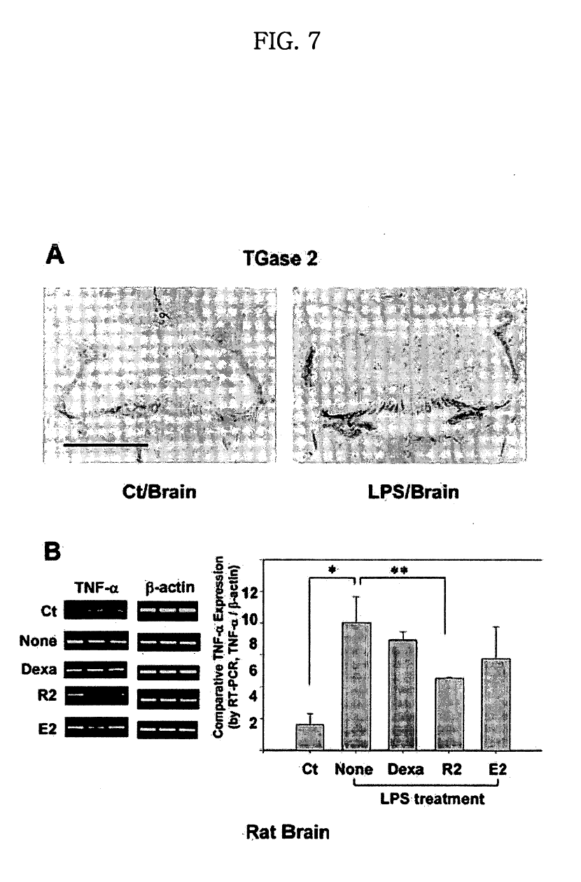

TGase 2 inhibitors were examined for effects on brain injuries induced in rats using LPS. Immunohistochemical staining analysis showed that TGase 2 expression increased in brains of the rats killed 1 hour after peritoneal injection of 2.5 mg/kg of LPS, compared with rats killed after peritoneal injection of saline alone (FIG. 7A). To examine the effect of TGase 2 inhibitors on neuroinflammation, TGase inhibitors were injected twice into the rat brain. The expression level of the inflammatory cytokine TNF-α was observed to be significantly reduced by the inhibitors as measured by RT-PCR with β-actin used as a control.

As described hereinbefore, a TGase 2 inhibitor or activator can be effectively detected by measuring the level of free or polymerized I-κBα, which is revealed to be a target of TGase 2, or the activation of NF-κB in accordance with the present invention.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

1. A method for screening for a Transglutaminase 2 (TGase 2) activator, comprising:
(a) treating a cell expressing I-κBα and NF-κB with a candidate activator of TGase 2;
(b) inducing the expression of TGase 2 in the cells; and
(c) comparing the level of free I-κBα, the level of polymerized I-κBα, or the activation of NF-κB of the cell treated with the candidate activator with the level of free I-κBα, the level of polymerized I-κBα, or the activation of NF-κB of a control not treated with the candidate activator
wherein a decrease in free I-κBα, an increase in the level of polymerized I-κBα, or an increase in the activation of NF-κB indicates the presence of the TGase 2 activator.

2. A method for screening for a Transglutaminase 2 (TGase 2) inhibitor, comprising:
(a) treating a cell expressing I-κBα and NF-κB with a candidate inhibitor of TGase 2;
(b) inducing the expression of TGase 2 in the cells; and
(c) comparing the level of free I-κBα, the level of polymerized I-κBα, or the activation of NF-κB of the cell treated with the candidate inhibitor with the level of free I-κBα, the level of polymerized I-κBα, or the activation of NF-κB of a control not treated with the candidate inhibitor
wherein an increase in free I-κBα, a decrease in the level of polymerized I-κBα, or a decrease in the activation of NF-κB indicates the presence of the TGase 2 inhibitor.

3. The method of claim 1 or 2, wherein steps (a) and (b) are performed simultaneously.

4. The method of claim 1 or 2, wherein the expression of TGase 2 is induced with a factor selected from a group consisting of LPS (lipopolysaccharide), UV light, ionizing radiation, glutamate, calcium ionophore, maitotoxin, RA (Retinoic acid), inflammation-induced cytokines, glutamate, oxidative stress, viral infection and combinations thereof.

5. The method of claim 1 or 2, wherein the level of free or polymerized I-κBα is detected using a specific antibody against I-κBα.

6. The method of claim 5, wherein the level of free or polymerized I-κBα is detected using Western blotting assay.

7. The method of claim 1 or 2, wherein the activation of NF-κB is detected using a reporter assay or an electrophoretic mobility shift assay.

8. A method for screening for a TGase 2 inhibitor or activator, comprising:
(a) treating isolated I-κBα with a candidate inhibitor or activator of TGase 2;
(b) treating the isolated I-κBα with isolated TGase 2; and
(c) detecting the level of free or polymerized I-κBα.

9. The method as claimed in claim 8, wherein steps (a) and (b) are performed simultaneously.

10. The method as claimed in claim 8, wherein the level of free or polymerized I-κBα is detected using a specific antibody against I-κBα.

11. The method as claimed in claim 10, wherein the level of free or polymerized I-κBα is detected using Western blotting assay.
